(19) United States
(12) Patent Application Publication
(10) Pub. No.: US 2019/0008370 A1
(43) Pub. Date: Jan. 10, 2019

(54) Medical Tubing Member

(71) Applicant: Olympus Corporation, Tokyo (JP)

(72) Inventor: Kazuhiko Hino, Tokyo (JP)

(73) Assignee: Olympus Corporation, Tokyo (JP)

(21) Appl. No.: 16/130,234

(22) Filed: Sep. 13, 2018

Related U.S. Application Data
(63) Continuation of application No. PCT/JP2017/005460, filed on Feb. 15, 2017.

(30) Foreign Application Priority Data
Mar. 16, 2016 (JP) 2016-052303

(51) Int. Cl. A61B 1/015; A61B 17/34; A61B 1/00

(52) U.S. Cl. CPC A61B 1/015; A61B 17/34; A61B 1/00131; A61B 1/00087; A61B 1/00094

(57) ABSTRACT

A medical tubing member is used in a medical system that allows fluid to flow between an inside and outside of a subject. The medical tubing member includes a first tubing member and a second tubing member. The first tubing member is configured to be arranged outside the subject and is configured to be connected to a first line disposed in an endoscope. The first tubing member is configured to supply the fluid from the outside of the subject to the inside of the subject. The second tubing member is configured to be arranged outside the subject and configured to be connected to a second tubular line disposed in the endoscope. The second tubing member is configured to discharge the fluid from the inside of the subject to the outside of the subject. The second tubing member is disposed inside the first tubing member.

### CROSS-REFERENCE TO RELATED APPLICATIONS

This application is a continuation application of PCT Application No. PCT/JP2017/005460 filed on Feb. 15, 2017, which in turn claim priority to the Japanese Patent Application No. 2016-52303 filed Mar. 16, 2016 in Japan which is hereby incorporated by reference in its entirety.

### TECHNICAL FIELD

The technology disclosed herein relates to a medical tubing member, and particularly to a medical tubing member useful when performing treatment to an affected part in a body and the like.

### DESCRIPTION OF THE RELATED ART

Treatment has been conventionally practiced to cauterize an affected part in the body while observing conditions of the affected part through an endoscope. For example, an endoscope that is interpreted to be usable for the above-described treatment is disclosed in Japanese Patent Laid-Open No. 1993-285094. Specifically, Japanese Patent Laid-Open No. 1993-285094 discloses a configuration that a laser probe is inserted in a surgical instrument channel disposed in an insertion section of an endoscope. The laser is irradiated on an affected part with the laser probe projecting at a tip portion thereof from an opening of the surgical instrument channel. The opening is on the side of a distal end of the surgical instrument channel. The affected part is cauterized by the laser. During the above-described treatment, a situation may arise such that the observation visual field of the endoscope would be impaired, for example, from vaporization of constituents derived from a biological tissue along with cauterization of the affected part. Japanese Patent Laid-Open No. 1993-285094 also discloses a configuration for responding to a situation similar to the above-described situation.

Specifically, Japanese Patent Laid-Open No. 1993-285094 discloses a configuration of a circulation device. The circulation device can circulate fluid, such as gas The circulation device includes a gas supply pump and a gas suction pump. The gas supply pump is connected to a gas supply line disposed in the insertion section of the endoscope. The gas suction pump is connected to a gas suction line disposed in the insertion section. The amount of supply gas upon supplying the gas to the gas supply line by the gas supply pump and the amount of suction gas upon suctioning the gas from the gas suction line by the gas suction pump are controlled to be the same for avoiding the impairment to the visual field due to smoke produced as a result of the laser cauterization. According to the configuration disclosed in Japanese Patent Laid-Open No. 1993-285094, however, there is a problem that the amount of supply gas to the gas supply line may become greater than the amount of suction gas from the gas suction line, for example, upon occurrence of damage to a gas suction tube used for the connection between the gas suction pump and the gas suction line. The configuration disclosed in Japanese Patent Laid-Open No. 1993-285094, therefore, involves the possibility that, upon conducting treatment to an affected part in the body of patient while allowing fluid to circulate between the inside and outside of the body, the treatment may not be smoothly conducted due to an oversupply of the fluid.

Accordingly, there is need for a medical tubing member that can address the aforementioned problem.

### BRIEF SUMMARY OF EMBODIMENTS

With the foregoing circumstances in view, the present disclosure has as an object thereof the provision of a medical tubing member that, upon conducting treatment to an affected part in the body while allowing fluid to circulate between the inside and outside of the body, can avoid an oversupply of the fluid into the body.

A medical tubing member according to an aspect of the present disclosure is a medical tubing member for use in a medical system that allows fluid to flow between an inside and outside of a subject. The medical tubing member includes a first tubing member and a second tubing member. The first tubing member is configured to be arranged outside the subject and is configured to be connected to a first line disposed in an endoscope. The endoscope is to be inserted into the subject. The first tubing member is configured to supply the fluid from the outside of the subject to the inside of the subject via the endoscope. The second tubing member is configured to be arranged outside the subject and is configured to be connected to a second tubular line disposed in the endoscope. The second tubing member is configured to discharge the fluid from the inside of the subject to the outside of the subject via the endoscope. The second tubing member is disposed inside the first tubing member.

A medical tubing member according to another aspect the present disclosure is a medical tubing member for use in a medical system that allows fluid to flow between an inside and outside of a subject. A first tubing member is configured to be arranged outside the subject, and configured to supply the fluid from the outside of the subject to the inside of the subject. A second tubing member is configured to be arranged outside the subject, and configured to discharge the fluid from the inside of the subject to the outside of the subject. A first connecting member is configured to connect the first tubing member to a first trocar to be pierced through a body wall of the subject. A second connecting member is configured to connect the second tubing member to a second trocar to be pierced through the body of the subject. The second tubing member is disposed inside the first tubing member.

A medical tubing member according to another aspect of the present disclosure is a medical system. The medical system includes an endoscope, a light source, a video processor, a circulating tubing member, and a circulation device. All of which configured to be attached to one another to perform treatment of an affected tissue of a body while controlling circulation of fluid between respective inside and outside of the body without and avoiding an oversupply of the fluid into the body during the treatment. A medical tubing member for use in the medical system permits fluid to flow between the respective inside and outside of the body. A first tubing member is configured to be arranged outside the body, and configured to be connected to a first tubular line disposed in the endoscope. The endoscope is to be inserted into the body. The first tubing member is configured to supply the fluid from the outside of the body to the inside of the body via the endoscope. A second tubing member is configured to be arranged outside the body, and configured to be connected to a second tubular line disposed in the endoscope and to discharge the fluid from the inside of the body to the outside of the body via the endoscope. The second tubing, member is disposed inside the first tubing member.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a front view illustrating essential parts of an example of a medical system in which a medical tubing member according to a first embodiment of the present disclosure is used.

FIG. 2 is a cross-sectional view taken along line II-II of FIG. 1.

FIG. 3 is a cross-sectional view illustrating an example of a specific configuration of a connecting part between an endoscope and the medical tubing member in the medical system of FIG. 1.

FIG. 4 is a view for describing a modification of the medical tubing member in FIG. 1.

FIG. 5 is a cross-sectional view for describing another modification of the medical tubing member in FIG. 1.

FIG. 6 is a cross-sectional view for describing a further modification of the medical tubing member in FIG. 1.

FIG. 7 is a cross-sectional view for describing yet another modification of the medical tubing member in FIG. 1.

FIG. 8 is a cross-sectional view for describing a yet further modification of the medical tubing member in FIG. 1.

FIG. 9 is a front view illustrating essential parts of another example of the medical system, in which a medical tubing member according to a second embodiment of the present invention is used.

FIG. 10 is a schematic view for describing a modification of a circulation device in the medical system of FIG. 1 or FIG. 9.

### DETAILED DESCRIPTION OF THE EMBODIMENTS

In the following description, various embodiments of the technology will be described. For purposes of explanation, specific configurations and details are set forth in order to provide a thorough understanding or the embodiments. However, it will also be apparent to one skilled in the art that the technology disclosed herein may be practiced without the specific details. Furthermore, well-known features may be omitted or simplified in order not to obscure the embodiment being described.

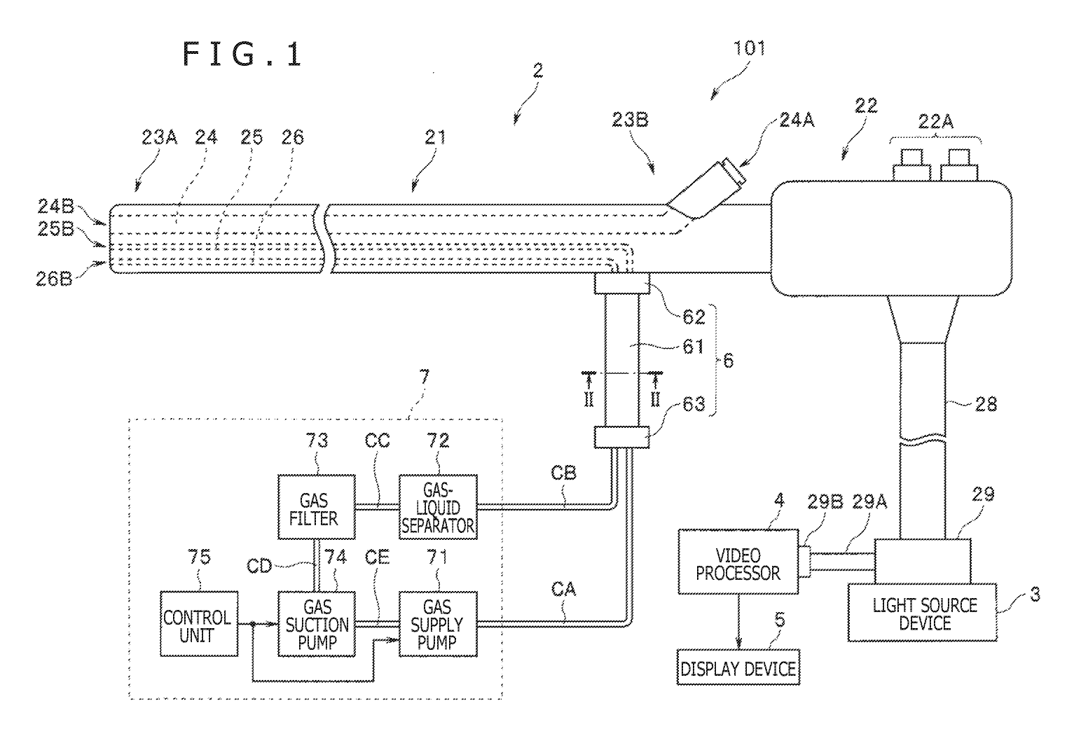

FIG. 1 through FIG. 8 relate to a first embodiment of the present invention. As illustrated in FIG. 1, a medical system 101 includes an endoscope 2, a light source device 3, a video processor 4, a display device 5, a circulation tubing member 6, and a circulation device 7. The endoscope 2 is configured to be insertable into the body of a subject. The endoscope is also configured to take an image of an object such as a biological tissue in the body and to output image signals. The light source device 3 is configured to supply illumination light via a light guide (not shown) inserted and disposed inside the endoscope 2. The illumination light is to be used for the observation of the object. The video processor 4 is configured to generate and output video signals or the like corresponding to the image signals outputted from the endoscope 2. The display device 5 is configured to display an observation image or the like corresponding to the video signals outputted from the video processor 4. FIG. 1 is a view illustrating some main parts of an example of a medical system.

The endoscope 2 includes an insertion section 21 and an operation unit 22. The insertion section 21 is formed in a slender shape to permit insertion into the body of the subject. The operation unit 22 is disposed on the side of a proximal end of the insertion section 21 opposite from the distal end of the insertion section 21. In addition, the endoscope 2 has a configuration so that it is detachably connected to the light source device 3 and video processor 4 via a universal cable 28 extending from the operation unit 22. Specifically, as illustrated by way of example in FIG. 1, the endoscope 2 is configured to be detachably connected to the light source device 3 via a universal connector 29 on an end portion of the universal cable 28. Further, as illustrated by way of example in FIG. 1, the endoscope 2 is also configured to be detachably connected to the video processor 4 via a connector 29B on an end portion of a signal cable 29A disposed extending from the universal connector 29. At a proximal end portion 23B of the insertion section 21, the endoscope 2 has a configuration that a connector 62 is detachably connected thereto. The connector 62 is disposed on the circulation tubing member 6.

The endoscope 2 is also configured to be detachably connected to the circulation device 7 via a gas supply tube CA and a gas suction tube CB that are connected to a connector 63 disposed on the circulation tubing member 6. In a distal end portion 23A of the insertion section 21, an illumination window (not shown) is disposed. Illumination light is emitted by way of the light guide of the endoscope 2. The illumination window is configured to have a lens or the like for irradiating illumination light on the object that exists in front of the distal end portion 23A. Also an observation window is disposed in the distal end portion 23A of the insertion section 21. The observation window includes lenses or the like for obtaining an optical image of the object by the illumination light from the illumination window. In the distal end portion 23A of the insertion section 21, an image sensor (not shown) such as charge coupled device (CCD) is also disposed. The image sensor is configured (i) to generate image signals by imaging the optical image of the object as obtained through the observation window and (ii) to output the thus-generated image signals to the video processor 4 via a signal line (now shown) accommodated in the endoscope 2.

In the proximal end portion 23B of the insertion section 21, a surgical instrument insertion opening 24A is disposed. This surgical instrument insertion opening 24A is formed as an opening through which a surgical instrument can be inserted. In addition, a forceps channel 24 is disposed inside the insertion section 21. The surgical instrument is inserted from the surgical instrument insertion opening 24A. This forceps channel 24 is formed as a tubular line through which the surgical instrument can be inserted. In the distal end portion 23A of the insertion section 21, a surgical instrument projection opening 24B is disposed. This surgical instrument projection opening 24B is formed as an opening through which the surgical instrument inserted inside the forceps channel 24 is allowed to project in front of the tip portion 23A. Inside the insertion section 21, a gas supply line 25 is disposed. This gas supply line 25 is formed as a tubular line which allows flowing of gas that is fluid. The fluid supplied from the circulation device 7 via the gas supply tube CA and circulation tubing member 6. In the distal end portion 23A of the insertion section 21, a gas supply port 25B is disposed. Gas flows inside the gas supply line 25. This gas supply port 25B is formed as an opening for discharging gas in front of the distal end portion 23A.

In the distal end portion 23A of the insertion section 21, a gas suction port 26B is disposed. This suction portion 26B is formed as an opening for suctioning gas, which exists around the distal end portion 23A, by the circulation device 7. Inside the insertion section 21, a gas suction line 26 is disposed. This gas suction line 26 is formed as a tubular line which allows gas, which is suctioned from the gas suction port 26B, to flow therethrough.

The operation unit 22 is configured having a shape that allows a user such as a surgeon to hold and operate the same. On the operation unit 22, a scope switch assembly 22A is disposed. This scope switch assembly 22A is configured including one or more switches that can perform an instruction to the video processor 4 according to input operation by the user. The light source device 3 is configured including, for example, a white light source so that white light is emitted from the white light source and the white light can be supplied as illumination light. The video processor 4 is configured so that, while the connector 29B is connected thereto, drive signals are generated and outputted to drive an imaging device of the endoscope 2. The video processor 4 is also configured so that, while the connector 29B is connected thereto, signal processing such as noise removal, white balance and gamma correction are applied to image signals outputted from the imaging device of the endoscope 2 to generate video signals and output the thus-generated video signals to the display device 5.

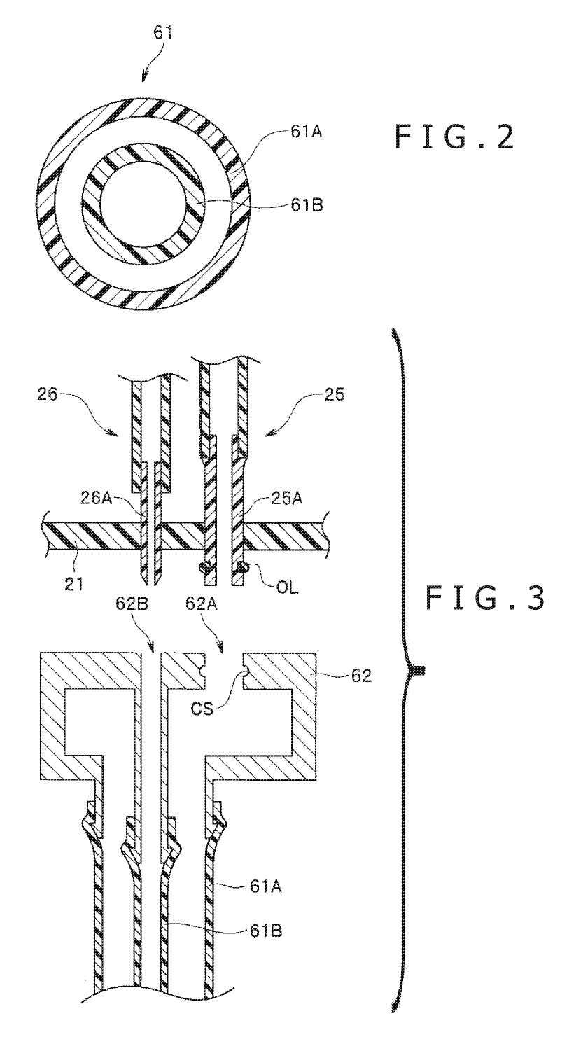

The circulation tubing member 6 is configured as a medical tubing member having a tubular body 61, the connector 62 disposed on the side of an end of the tubular body 61, and the connector 63 disposed on the side of an opposite end of the tubular body 61. As illustrated by way of example in FIG. 2, the tubular body 61 is configured including a gas supply tubing member 61A and a gas suction tubing member 61B. This gas supply tubing member 61A allows gas, which is supplied via the gas supply tube CA connected to the connector 63, to flow to the gas supply line 25. On the other hand, the gas suction tubing member 61B allows gas, which is suctioned via the gas suction line 26, to flow to the gas suction tube CB connected to the connector 63. The tubular body 61 is also configured so that at least a part thereof is arranged outside the subject. FIG. 2 is a cross-sectional view taken along line II-II of FIG. 1. The gas supply tubing member 61A is formed, for example, from a flexible material, such as a resin. On the other hand, the gas supply tubing member 61A is formed, for example, as a hollow tube member having a circular cross-section as illustrated in FIG. 2. The gas suction tubing member 61B is formed, for example, from a flexible material, such as a resin. Further as illustrated lay of example in FIG. 2, the gas suction tubing member 61B is disposed inside the gas supply tubing member 61A, and is formed as a hollow tubular body having a circular cross-section.

According to the configuration as described hereinbefore, no wall is used in common in the gas supply tubing member 61A and the gas suction tubing member 61B. In other words, the outer wall of the gas suction tubing member 61B does not make up the inner wall of the gas supply tubing member 61A. As illustrated by way example in FIG. 3, the connector 62 is configured including an opening 62A and an opening 62B. The opening 62A communicates to the side of an outlet of the gas supply tubing member 61A. The opening 62B communicates to the side of an inlet of the gas suction tubing member 61B. FIG. 3 is a view illustrating an example of a specific configuration of a connecting part between the endoscope and the circulation tubing member in the medical system of FIG. 1.

As illustrated by way of example in FIG. 3, the connector 62 is configured so that the gas supply tubing member 61A can be connected at the opening 62A thereof to a connection port 25A of the gas supply line 25, the connection port 25A being disposed while being exposed to the outside from the proximal end portion 23B, and the gas suction tubing member 61B can be connected at the opening 62B thereof to a connection port 26A of the gas suction line 26, the connection port 26A being disposed while being exposed to the outside from the proximal end port on 23B.

In an inner circumferential wall of the opening 62A of the connector 62, an annular groove portion CS is disposed. This annular groove portion CS can be fitted on an O-ring OL disposed on an outer circumferential wall of the connection port 25A. The opening 62B of the connector 62 is formed having an inner diameter, which is smaller than the inner diameter of the opening 62A and is substantially equal to the outer diameter of the connection port 26A. According the configuration as described hereinbefore, if the connector 62 is properly connected to the proximal end portion 23B of the insertion section 21, the O-ring OL of the connection port 25A is fittedly disposed in the groove portion CS of the opening 62A, and the connection port 26A is fittedly disposed in the opening 62B. According the configuration as described hereinbefore, connection of the connection port 25A to the opening 62B can be avoided by the O-ring OL. According the configuration as described hereinbefore, it is, therefore, possible to avoid a situation in which the opening 62A would be connected to the connection port 26A and the opening 62B would be connected to the connection port 25A, in other words, a situation in which the connector 62 would be connected in an opposite direction to the proximal end portion 23B of the insertion section 21.

The connector 63 is configured to allow the gas supply tube CA, which connected to the circulation device 7, to communicate to the side of an inlet of the gas supply tubing member 61A at an outlet-side end portion of the gas supply tube CA. The connector 63 is also configured to allow the gas suction tube CB, which is connected to the circulation device 1, to communicate to the side of an outlet of the gas suction tubing member 61B at an inlet-side end portion of the gas suction tube CB. Although not illustrated in any drawing, the connector 63 is configured (i) to have two connection ports formed in shapes different from each other, (ii) to be connectable to the gas supply tube CA, more specifically to the outlet-side end portion of the gas supply tube CA at only one of the two connection ports, and (iii) to be connectable to the gas suction tube CB, more specifically to the inlet-side end portion of the gas suction tube CB at only the other connection port of the two connection port. The two connection ports of the connector 63 may be, for example, those configured by changing the shapes of the above-described connection ports 25A and 26A as desired. The circulation device 7 is configured as a device for allowing gas to circulate between the inside and outside of the subject. In other words, the circulation device 7 is configured as a device that allows gas or fluid to flow between the inside of the subject and the outside of the subject. Specifically, as illustrated by way of example in FIG. 1, the circulation device 7 is configured including a gas supply pump 71, a gas-liquid separator 72, a gas filter 73, a gas suction pump 74, and a control unit 75. The gas-liquid separator 72 is detachably connected to the connector 63 via the gas supply tube CA. The gas-liquid separator 72 is detachably connected to the connector 63 via the gas suction tube CB. The gas filter 73 communicates to the gas-liquid separator 72 via a tubular line CC. The gas suction pump 74 communicates to the gas filter 73 via a tubular line CD. The gas supply pump 71 is configured to perform, responsive to control by the control unit 75, (i) operation to suction gas discharged from the gas suction pump 74 to the inside of the tubular line CE and also (ii) operation to supply the thus-suctioned gas to the gas supply line 25.

The gas-liquid separator 72 is configured to remove misty liquid from the fluid or the gas flowed in via the gas suction tube CB. The gas liquid separator 72 may be configured to remove solid contained in the liquid from the gas or the fluid flowed in via the gas suction tube CB. The gas-liquid separator 72 is also flow-out of the gas, from which the liquid is removed, to the tubular line CC. The gas filter 73 is configured to enable filtration of the gas, which has flowed in via the tubular line CC, and flowed-out of the filtered gas to the tubular line CD. The gas suction pump 74 is configured to perform, responsive to control by the control unit 75, (i) operation to suction gas, which exists around the distal end portion 23A, from the gas suction port 26B or to discharge the gas from the inside to the outside of the subject and also (ii) operation to discharge the suctioned gas. According to such operation of the gas suction pump 74, the gas is suctioned from the gas suction port 26B and the gas is purified through the gas-liquid separator 72 and gas filter 73. The purified gas is suctioned from the tubular line CD and is discharged to the inside of the tubular line CE.

The control unit 75 is configured to perform control on the gas supply pump 71 and the gas suction pump 74 to make equal or substantially equal the amount of gas to be supplied to the endoscope 2 per preset unit time and the amount of gas to be suctioned from the endoscope 2 per the preset unit time. In other words, the control unit 75 is configured to (i) perform control on the gas supply pump 71 to supply gas to the endoscope 2 only in a predetermined amount per preset unit time and also (ii) perform control on the gas suction pump 74 to suction gas from the endoscope 2 only in the predetermined amount per the preset unit time. Moreover, the control unit 75 is configured to perform control so that gas or fluid circulates between the inside and outside of the subject. The control unit 75 may be configured to perform control on the gas supply pump 71 and the gas suction pump 74 alternately, or may be configured to perform control on the gas supply pump 71 and the gas suction pump 74 simultaneously. A description will next be made about operations and functions of the medical system 101 of FIG. 1.

The use connects the individual parts of the medical system 101 and turns on the power, and then progressively inserts the insertion section 21 into the body of the subject while checking observation images to be displayed on the display 5. The user then places the distal end portion 23A at a desired position in the vicinity of an affected part in the body of the subject. The user performs treatment on the affected part with the surgical instrument, which is inserted in the forceps channel 24, being maintained projecting at a tip portion thereof from the surgical instrument projection opening 24B.

According to the user's operation as described hereinbefore, circulation of gas is performed between the inside and outside the subject. Specifically, the circulation is performed by a gas supply flow passage and a gas suction flow passage according to the user's operation requirement. Gas is supplied from the circulation device 7 outside the body of the subject. Through the gas supply flow passage, the gas is discharged into the body of the subject via the gas supply tube CA, the gas supply tubing member 61A and the gas supply line 25. On the other hand, gas is suctioned from the inside of the body of the subject. Through the gas suction flow passage, the gas is allowed to flow into the circulation device 7 outside the body of the subject via the gas suction line 26, the gas suction tubing member 61B and the gas suction tube CB. In other words, the gas supply tubing member 61A forms, in the outside of the body of the subject, a part of the flow passage for gas to be supplied into the body of the subject by the gas supply pump 71 of the circulation device 7. On the other hand, the gas suction tubing member 61B forms a part of the flow passage for gas to be suctioned from the body of the subject by the gas suction pump 74 of the circulation device 7. With the gas suction tubing member 61B disposed inside the gas supply tubing member 61A in this embodiment, the gas suction tubing member 61B is protected from damage as much as possible during the period that the circulation of gas is conducted between the inside and outside of the body of the subject.

According to this embodiment, a portion of gas is flowing inside the gas supply tubing member 61A (rather than the gas existing in the outside of the body) and is suctioned by the gas suction pump 74 even if the gas suction tubing member 61B is damaged during the period that the circulation of gas is conducted between the inside and outside of the body of the subject. Upon performing treatment of the affected part in the body while circulating gas between the inside and outside'of the body, this embodiment, therefore, makes it possible to avoid an oversupply of the gas into the body. The gas supply tubing member 61A and gas suction tubing member 61B in this embodiment may also be formed to have mutually-different ultimate tensile strengths, for example, against a tensile stress as a stress applied a direction in which the circulation tubing member 6 is pulled at opposite ends thereof from the outside. As another alternative, the gas supply tubing member 61A and gas suction tubing member 61B in this embodiment may also be formed to have mutually-different ultimate flexural strengths, for example, against a bending stress as a stress applied from the outside in a direction in which the linear circulation tubing member 6 is bent.

Now assume, for example, that circulation tubing member 6 is formed with the gas supply tubing member 61A having lower ultimate tensile strength than the gas suction tubing member 61B against a tensile stress. When the circulation tubing member 6 is pulled, the gas supply tubing member 61A is broken first and at the same time, gas leaks out to the outside from the broken portion of the gas supply tubing member 61A. If the circulation tubing member 6 is formed with the gas supply tubing member 61A having lower ultimate tensile strength than the gas suction tubing member 61B against a tensile stress, the user can, therefore, be facilitated to recognize breakage of the circulation tubing member 6. Also assume, for example, that the circulation tubing member 6 is formed with the gas suction tubing member 61B having lower ultimate tensile strength than the gas supply tubing member 61A against a tensile stress. When the circulation tubing member 6 is pulled, the gas suction tubing member 61B is broken first and at the same time, a portion of gas which is flowing inside the gas supply tubing member 61A flows in from the broken portion of the gas suction tubing member 61B and is suctioned by the gas suction pump 74. If the circulation tubing member 6 is formed with the gas suction tubing member 61B having lower ultimate tensile strength than the gas supply tubing member 61A to a tensile stress, it is, therefore, possible to avoid a situation that the pressure inside the body of the subject would excessively increase or decrease following breakage of the gas suction tubing member 61B.

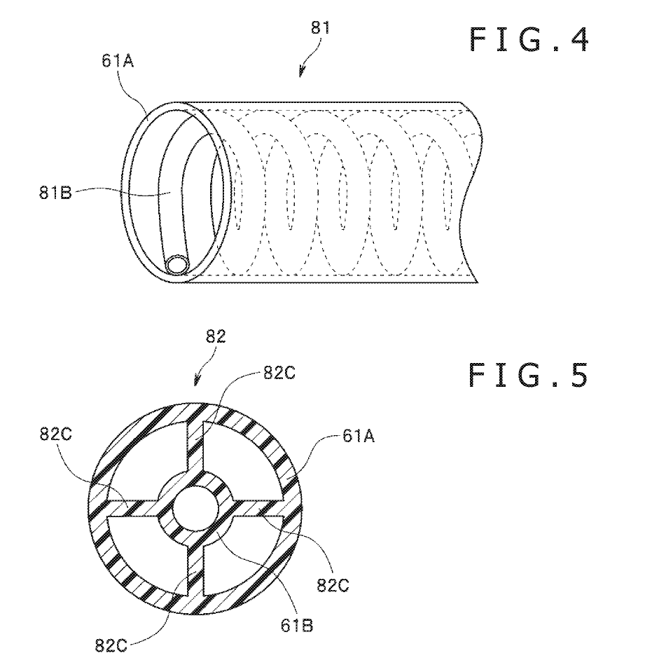

As an alternative, the circulation tubing member 6 in this embodiment may also be configured including, for example, a tubular body 81 illustrated in FIG. 4 in place of the tubular body 61 illustrated in FIG. 2.

FIG. 4 is a view for describing a modification of the circulation tubing member in the medical system of FIG. 1. Specifically, the tubular body 81 is formed by disposing a gas suction tubing member 81B, which is formed helically, inside the gas supply tubing member 61A. According to the configuration of the tubular body 81 as described hereinbefore, even if a relatively large bending stress is applied to the circulation tubing member 6, for example, the flow rates of gas that is flowing through the gas supply tubing member 61A and gas suction tubing member 81B remain practically unchanged so that the circulation of gas by the circulation device 7 can be stabilized. The tubular body 81 is not limited to one formed with the single helical gas suction tubing member 81B disposed inside the gas supply tubing member 61A, but may also be formed by disposing two or more helical gas suction tubing members 81B inside the gas supply tubing member 61A. The tubular body 81 is formed, for example, by including two gas suction tubing members 81B and forming a double helix with the two gas suction tubing members 81B inside the gas supply tubing member 61A.

As another alternative, the circulation tubing member 6 in this embodiment may also be configured, for example, including a tubular body 82 as illustrated in FIG. 5 instead of the tubular body 61 as illustrated in FIG. 2.

FIG. 5 is a view for describing another modification of the circulation tubing member in the medical system of FIG. 1. Specifically, the tubular body 82 is formed, as illustrated in FIG. 5, with four support members 82C disposed in at least an inner part of the gas supply tubing member 61A. The four support members 82C are configured to arrange the gas suction tubing member 61B at a preset position inside the gas supply tubing member 61A by supporting the gas suction tubing member 61B from four directions (up, down, left and right). The support members 82C are formed so that an inner circumferential wall of the gas supply tubing member 61A and an outer circumferential wall of the gas suction tubing member 61B are integrally connected at parts thereof together. Further, the support members 82C are also configured, for example, so that in the gas supply tubing member 61A, the gas suction tubing member 61B is arranged at a position where the gas supply tubing member 61A and gas suction tubing member 61B have coinciding central axes. According the configuration of the tubular body 82 as described hereinbefore, the gas flow passage is formed by the gas suction tubing member 61B. The gas flow passage can be prevented as much as possible from being constricted as a result of an increase in the internal pressure of the gas supply tubing member 61A and/or a decrease in the internal pressure of the gas suction tubing member 61B.

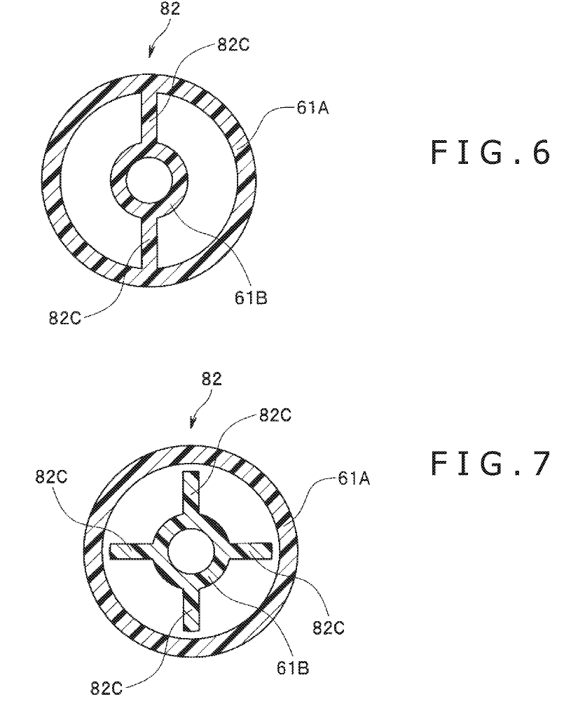

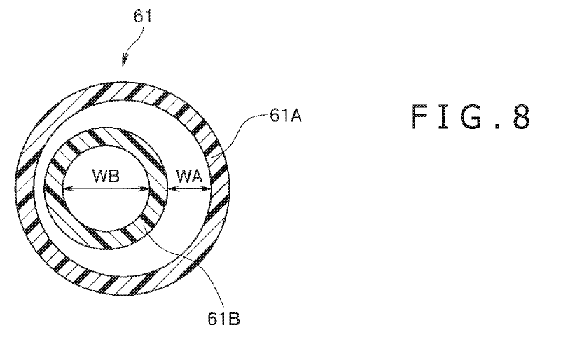

The tubular body 82 is not limited to that formed including the four support members 82C, and as illustrated by way of example in FIG. 6, may be formed including two support members 82C for supporting the gas suction tubing member 61B in up and down or left and right, two directions. The support members 82C in the tubular body 82 are not limited to those formed as described hereinbefore, but as illustrated by way of example in FIG. 7, may be formed integrally with a part of the outer circumferential wall of the gas suction tubing member 61B but may be formed as discrete members from the inner peripheral wall of the gas supply tubing member 61A. FIGS. 6 and 7 are views for describing further modifications of the circulation tubing member the medical system of FIG. 1. Further, in view of a situation that solid soil may be suctioned from the gas suction port 26B, the tubular body 61 in the first embodiment may also be configured so that as illustrated by way of example in FIG. 8, the gas suction tubing member 61B allows solid soil to pass therethrough and the solid soil has a maximum width WB greater than that WA of solid soil which the gas supply tubing member 61A allows to pass therethrough. The solid soil is derived from a biological tissue, for example, such as residues or the like occurred by treatment with the endoscope 2. FIG. 8 is a view for describing a yet further modification of the circulation tubing member in the medical system of FIG. 1. The tubular body 61 is not limited to the configuration as illustrated in FIG. 8, but may also be configured so that the gas suction tubing member 61B has a greater cross-sectional area than the gas supply tubing member 61A. According to the configuration as described hereinbefore, it is possible to make, for example, semi-solid soil, which is suctioned by the gas suction pump 74, hardly clog in the gas suction tubing member 61B.

By modifying the configuration of the circulation device 7 or the like as needed, for example, this embodiment can also be applied in substantially the same manner to a case where liquid such as physiological saline is circulated between the inside and outside of the body of a subject. In other words, this embodiment can be applied to a case where fluid, that is, gas or liquid is circulated between the inside and outside of the body of a subject. The endoscope 2 in this embodiment, insofar as it is for use in the body of a subject, may be one to be used in the digestive organs such as the stomach and large intestine, or may be one to be used in parts other than the digestive organs, such as the urinary organs and the like.

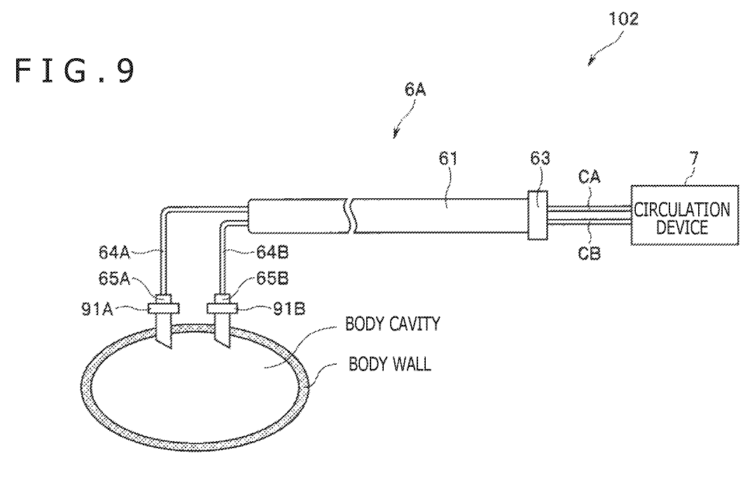

FIG. 9 relates to a second embodiment of the present disclosure. In this embodiment, a detailed description on elements having similar configurations or the like as the corresponding ones in the first embodiment is not repeated again to avoid redundancy, and a description will be made primarily on elements having configurations or the like different from the corresponding ones in the first embodiment. A medical system 102 for use with this embodiment is configured as a system usable when performing treatment of an affected part in the body or in the subject by using a laparoscope (now shown). Specifically, the medical system 102 includes, as illustrated by way of example in FIG. 9, a circulation tubing member 6A and the circulation device 7. The circulation tubing member 6A is configured to communicate to the inside of the body of a subject via two trocar 91A and 91B pierced through a body wall of the subject. The circulation device 7 is configured to be detachably connected to the circulation tubing member 6A via the gas supply tube CA and gas suction tube CB. FIG. 9 is a view illustrating main parts of another example of a medical system in which a medical tubing member according to the second embodiment of the present invention is used. The circulation tubing member 6A is configured including the tubular body 61, the connector 63, and a gas supply tube 64A and gas suction tube 64B. The connector 63 is disposed on the side of the proximal end of the tubular body 61. The gas supply tube 64A and gas suction tube 64B are disposed extending, from the side of the distal end of the tubular body 61. The gas supply tube 64A is at an end portion on an inlet side thereof in communication with the gas supply tubing member 61A of the tubular body 61. A gas supply connector 65A is disposed on an end portion on an outlet side of the gas supply tube 64A. The gas supply connector 65A can be connected to an opening of the trocar 91A to be pierced through the body wall of the subject. A gas suction connector 65B is disposed on an end portion on an inlet side of the gas suction tube 64B. The gas suction connector 65B can be connected to an opening of the trocar 91B to be pierced through the body wall of the subject. The gas suction tube 64B is at an end portion on an outlet side thereof in communication with the gas suction tubing member 61B of the tubular body 61.

In other words, the circulation tubing member 6A is configured including the gas supply tube 64A and gas supply connector 65A, which are connecting members configured to connect the gas supply tubing member 61A to the trocar 91A to be pierced through the body wall of the subject. In addition, the circulation tubing member 6A is also configured including the gas suction tube 64B and gas suction connector 65B, which are connecting members configured to connect the gas suction tubing member 61B to the trocar 91B to be pierced through the body wall of the subject. According to the configuration of this embodiment as described hereinbefore, circulation is performed by a gas supply flow passage and a gas suction flow passage. Gas is supplied from the circulation device 7 outside the body of the subject.

The gas is discharged to the inside of the subject via the gas supply tube CA, the gas supply tubing member 61A, gas supply tube 64A and trocar 91A through the gas suction flow passage. Gas is suctioned from the inside of the body of the subject. The gas flows into the circulation device 7 outside the body of the subject via the trocar 91B, the gas suction tube 64B, the gas suction tubing member 61B and the gas suction tube CB through the gas suction flow passage. Therefore, upon performing treatment of the affected part the body while circulating gas between the inside and outside of the body, this embodiment also makes it possible to avoid an oversupply of the gas into the body as in the first embodiment.

According to the configuration of this embodiment, it is also possible to improve the degrees of freedom in the positions of arrangement of the subject and the circulation device 7 compared with the first embodiment.

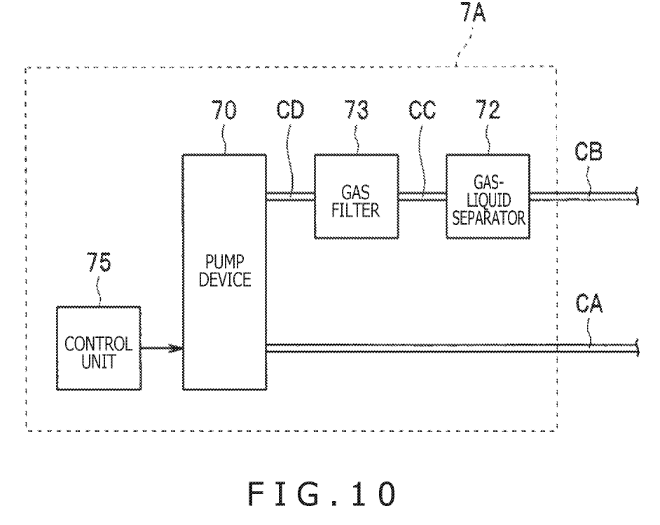

In each of the embodiments described hereinbefore, the medical system 101 or 102 may also be configured using a circulation device 7A as illustrated by way of example in FIG. 10 instead of the circulation device 7 in which the gas supply pump 71 and the gas suction pump 74 are disposed discretely. FIG. 10 is a view for describing the modification of the circulation device in the medical system of FIG. 1 or FIG. 9. The circulation device 7A is configured including a pump device 70, the gas-liquid separator 72, the gas filter 73, and the control unit 75. The pump device 70 is configured to be detachably connected to the connector 63 via the gas supply tube CA and also to communicate to the gas filter 73 via the tubular line CD disposed in the inside of the circulation device 7A. The pump device 70 is configured including a single pump having the functions of the gas supply pump 71 and the gas suction pump 74 in combination. Described specifically, the pump device 70 is configured to perform, responsive to control by the control unit 75, (i) operation to suction gas, which exists around the distal end portion 23A, from the gas suction port 26B and also (ii) operation to supply the suctioned gas to the gas supply line 25. According to such operation of the pump device 70, the gas suctioned from the gas suction port 26B is purified through the gas-liquid separator 72 and gas filter 73, and the thus-purified gas is suctioned from the tubular line CD and is discharged to the inside of the gas supply tube CA.

While various embodiments of the disclosed technology have been described above, it should be understood that they have been presented way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. A medical tubing member for use in a medical system that allows fluid to flow between an inside and outside of a subject, comprising:
a first tubing member configured to be arranged outside the subject, and configured to be connected to a first tubular line disposed in an endoscope, which is to be inserted into the subject, and to supply the fluid from the outside of the subject to the inside of the subject via the endoscope; and
a second tubing member configured to be arranged outside the subject, and configured to be connected to a second tubular line disposed in the endoscope and to discharge the fluid from the inside of the subject to the outside of the subject via the endoscope;
wherein the second tubing member is disposed inside the first tubing member.

2. The medical tubing member of claim 1, further comprising:
a connector configured to permit connection of the first tubing member to the first tubular line and also connection of the second tubing member to the second tubular line.

3. The medical tubing member of claim 1, wherein
the first tubing member is at an end thereof in communication with a device that supplies the fluid to the inside of the subject, and also is at an opposite end thereof in communication with the first tubular line disposed in the endoscope to be inserted into the subject, and
the second tubing member is at an end thereof in communication with a device that discharges the fluid from the inside of the subject, and also is at an opposite end thereof in communication with the second tubular line disposed in the endoscope to be inserted into the subject.

4. The medical tubing member of claim 3, wherein
the medical system includes a circulation device having a pump configured to allow circulation of the fluid between the inside and outside of the subject via the endoscope,
the first tubing member is configured to form a part of a flow passage through which the fluid is supplied to the inside of the subject by the pump of the circulation device, and
the second tubing member is configured to form a part of a flow passage through which the fluid is discharged to the outside of the subject by the pump of the circulation device.

5. The medical tubing member of claim 1, wherein the first tubing member and the second tubing member are configured in a state that no wall is used in common.

6. The medical tubing member of claim 5, further comprising:
a support member configured to support the second tubing member inside the first tubing member so that the second tubing member is arranged at a preset position inside the first tubing member.

7. The medical tubing member of claim 6, wherein the support member is configured to arrange the second tubing member at a position inside the first tubing member where the first tubing member and second tubing member have coinciding central axes.

8. The medical tubing member of claim 6, wherein the support member is formed integrally on a part of an outer circumferential wall of the second tubing member, but is formed discretely from an inner circumferential wall of the first tubing member.

9. The medical tubing member of claim 6, wherein the support member configured to be formed so that the first tubing member and the second tubing member are integrally connected together at parts of inner circumferential wall of the first tubing member and outer circumferential wall of the second tubing member.

10. The medical tubing member of claim 1, wherein the first tubing member and second tubing member are each formed with a material having flexibility, and are configured to be formed so that they have mutually-different ultimate strengths to a stress applied externally.

11. The medical tubing member of claim 10, wherein the first tubing member is lower in the ultimate strength to the stress than the second tubing member.

12. The medical tubing member of claim 10, wherein the second tubing member is lower in the ultimate strength to the stress than the first tubing member.

13. The medical tubing member of claim 1, wherein the second tubing member is formed helically.

14. The medical tubing member of claim 1, wherein the second tubing member and first tubing member are configured so that the second tubing member allows solid soil to pass therethrough and the solid soil has a maximum width greater than that of solid soil which the first tubing member allows to pass therethrough.

15. A medical tubing member for use in a medical system that allows fluid to flow between an inside and outside of a subject, comprising:
a first tubing member configured to be arranged outside the subject, and configured to supply the fluid from the outside of the subject to the inside of the subject;
a second tubing member configured to be arranged outside the subject, and configured to discharge the fluid from the inside of the subject to the outside of the subject;
a first connecting member configured to connect the first tubing member to a first trocar to be pierced through a body wall of the subject; and
a second connecting member configured to connect the second tubing member to a second trocar to be pierced through the body wall of the subject,
wherein the second tubing member is disposed inside the first tubing member.

16. A medical system comprising:
an endoscope, a light source, a video processor, a circulating tubing member, and a circulation device all of which configured to be attached to one another to perform treatment of an affected tissue of a body while controlling circulation of fluid between respective inside and outside of the body without and avoiding an oversupply of the fluid into the body during the treatment;
a medical tubing member for use in the medical system that permits fluid to flow between the respective inside and outside of the body having
a first tubing member configured to be arranged outside the body, and configured to be connected to a first tubular line disposed in the endoscope, which is to be inserted into the body, and to supply the fluid from the outside of the body to the inside of the body via the endoscope, and
a second tubing member configured to be arranged outside the body, and configured to be connected to a second tubular line disposed in the endoscope and to discharge the fluid from the inside of the body to the outside of the body via the endoscope;
wherein the second tubing member is disposed inside the first tubing member.
